(19) United States
(12) Patent Application Publication
(10) Pub. No.: US 2003/0192075 A1
(43) Pub. Date: Oct. 9, 2003

(54) Elicitor from cladosporium

(76) Inventors: Frank Takken, Amsterdam (NL); Pierre De Wit, Wageningen (NL)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/332,059

(22) PCT Filed: Jul. 2, 2001
(86) PCT No.: PCT/EP01/07621

(30) Foreign Application Priority Data
Jul. 3, 2000 (EP) 00202320.8

(51) Int. Cl. A01H 1/00; C12N 15/82; C07H 21/04; C12P 21/02; C07K 14/415; C12N 5/04

(52) U.S. Cl. 800/279; 435/69.1; 435/320.1; 435/419; 536/23.2; 530/370; 800/294

(57) ABSTRACT

A novel transcription factor protein of Cladosporium fulvum which causes a hypersensitive response in plants and methods of using this protein for conferring pathogen resistance to plants are disclosed.

### BACKGROUND

Plants resistant to pathogens often are found to evoke their resistance through a mechanism which eventually yields a hypersensitive response (HR) resulting in rapid cell death of the infected plant cells. This rapid cell death or necrosis inhibits the pathogen from further growth and thus stops the infection. This mechanism is known already for a long time (Klement, Z., In: Phytopathogenic Prokaryotes, Vol. 2, eds.: Mount, M. S. and Lacy, G. H., New York, Academic Press, 1982, pp. 149-177). The HR is often confused with other lesion-like phenomena, but a typical HR gives local cell death and is associated with secondary responses such as callus deposition, generation of active oxygen species, induction of phytoalexins, changes in ion fluxes across membranes and induction of acquired resistance (AR) (Hammond-Kosack, K. E., et al., Plant Physiol. 110, 1381-1394, 1996).

The pathogen resistance is elicited by response to elicitor compounds, which are frequently found to be of proteinaceous nature (Arlat, M., et al., EMBO J., 13, 543-553, 1994; Baker, C. J. et al., Plant Physiol. 102, 1341-1344, 1993; Staskawicz, B. J. et al., Proc. Natl. Acad. Sci. USA 81, 6024-6028, 1984; Vivian, A. et al., Physiol. Mol. Plant Pathol. 35, 335-344, 1989; Keen, N. T., Ann. Rev. Gen. 24, 447-463, 1990; Ronald, P. C. et al., J. Bacteliol. 174, 1604-1611, 1992; Whitham, S. et al., Cell 78, 1-20, 1994;Kobe, B. and Deisenhofer, J., Trends Biochem. Sci. 19, 415, 1994; and Honée G. et al., Plant Mol. Biol. 29, 909-920, 1995). These elicitor proteins (encoded by avirulence genes) are produced by the pathogen and are thought to interact with a resistance protein available in the plant, therewith starting a cascade of events resulting in the HR-response. The elicitor proteins are characterized by that they are (race-)specific and only are able to elicit the response with a corresponding (also specific) resistance protein. The concept of avirulence-gene based resistance is also known under the name of the gene-for-gene response. Avirulence genes have been cloned from bacterial pathogens (such as Pseudomonas and Xanthomonas) and from fungal pathogens (such as Cladosporium fulvum, Rhynchosporium secalis and Phytophthora parasitica). Also plant genes coding for some of the corresponding resistance genes have been cloned (such as the tomato gene Cf9 corresponding to the avirulence gene avr9 fronm Cladosporium fulvum, and the tomato Pto-gene corresponding to the aviruience gene avrPto from Pseudomonas).

In recent years, a number of extracellular elicitor proteins of Cladosporium fulvum has been identified (Knogge, 1996, Lauge and De Wit, 1998). Cladosporium fulvum is a fungus which in nature is able to infect tomato plants. Corresponding with the elicitor molecules from the fungus there are a number of resistance proteins found, on basis of specific interactions between the diverse pathotypes of the fungus with the corresponding diverse varieties of tomato.

Methods to use resistance genes to confer pathogen resistance to plants are often hampered by the fact that the resistance is only limited to a few specific pathotypes. Further, it often appears that after triggering with an elicitor molecule the hypersensitive response can be rather slow and cannot prevent infection with rapidly progressing pathogens.

Thus there is still need for a system which can convey a fast and general pathogen resistance to plants upon start of infection and which does not switch on pathogen resistance when no pathogens are infecting.

### SUMMARY OF THE INVENTION

The invention now provides a method for the induction of pathogen resistance in plants characterized by transforming a plant with a polynucleotide *sequence comprising a pathogen inducible promoter which regulates the expression of a Cladosporium transcription factor protein comprising an amino acid sequence as depicted in SEQ ID NO: 2 or a mutein thereof which when constitutively expressed gives rise to a hypersensitive response in plants. A specific embodiment of the invention is such a method wherein the Cladosporium transcription factor is a peptide of 259 amino acids, as depicted in SEQ ID NO:2.

Next to a method for making plants resistant to pathogens also the protein itself, muteins thereof and the nucleotide sequence encoding the protein or its muteins form part of the invention.

Also part of the invention are plants made resistant against plant pathogens through any of the methods described.

### LEGENDS TO THE FIGS.

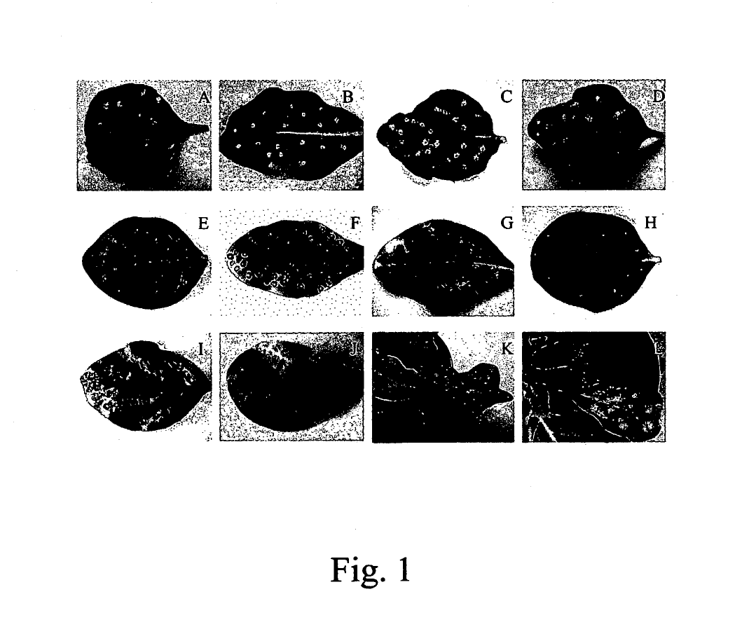

FIG. 1 Symptom of leaves toothpick inoculated with Agrobacterium containing pSfinx:: 43-7G (A-J, L) or pSfinx::Avr4 (K). A: N. benthamiana, B: N. clevelandii, C: N. glutinosa, D: N. cordifolia, E: N. rustica, F: N. langsdorfii, G: N. tobacum (cv. Samsun), I: N. paniculata, J: N. sylvestris, K and L: N. clevelandii.

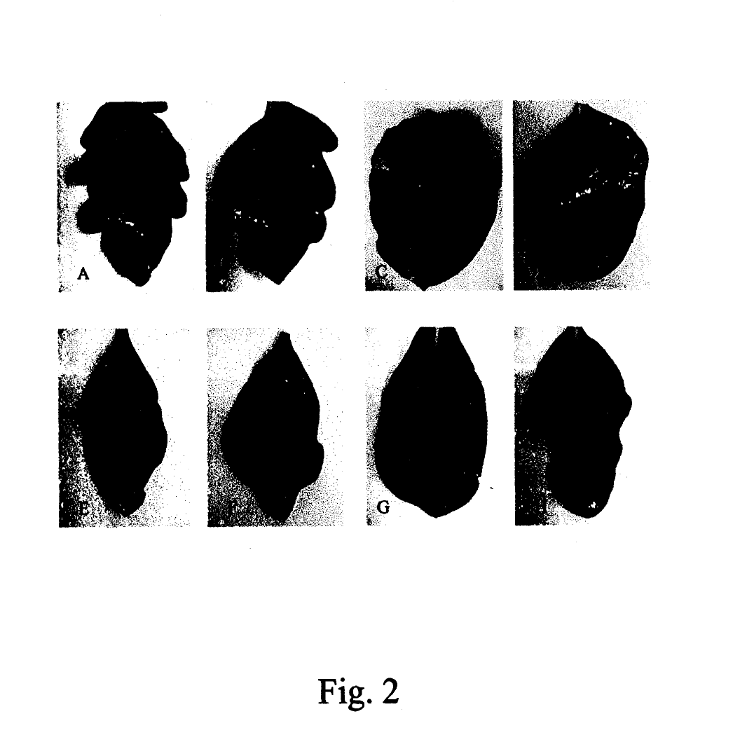

FIG. 2 Symptom of plants toothpick inoculated with Agrobacterium transformed with plasmids containing 5′ deletion constructs. Left side of tomato (A,B) and tobacco (C, D) leaves were inoculated with Agrobacterium transformed with plasmids containing the full length DNA, while the right side was inoculated with deletion Δ79 (A,C) or deletion Δ256 cDNA (B, D). The entire leaf of Nicotiana clevelandii (E-H) was inoculated with Agrobacterium containing a plasmid with the full length DNA (E), deletion Δ79 (F) or Δ256 (G). H shows the mosaic symptom of an uninoculated leaf of N. clevelandii plants that had been inoculated with Agrobacterium transformed with plasmids containing the 5′ deletion constructs.

### DETAILED DESCRIPTION

Surprisingly now a protein produced by Cladosporium fulvum has been found which is capable of giving an induction of the HR response The experiments reported in the experimental section show that the protein is able to elicit a response in both Cf4- and Cf9-containing tomato plants, and in a large number of tobacco species, indicating that the protein is not of the same type as the Avr-proteins (like Avr4 and Avr9) which cause a clear pathogen-host specificity. Further, from the molecular data disclosed in this application it can be derived that the protein of the invention is dissimilar to the Avr-proteins in a second way: it is a putative transcription factor (containing a bZIP motif sequence). The suspected mode of action is that, when expressed in a plant cell, this protein triggers the hypersensitive response through ectopic expression of genes involved in the execution of this defense reaction. Although it may thus not be a true ‘elicitor’ in the way this word is conventionally used, for ease of reference the term Cladosporium elicitor is used throughout this specification. It is well known to those skilled in the art that the hypersensitive defense response is an active response. This is clearly illustrated by the fact that protein elicitor-mediated induction of the hypersensitive response can be inhibited by alpha-amanitin, a powerful inhibitor of eukaryotic RNA polymerase, or by cycloheximide, a known inhibitor of eukaryotic protein synthesis (He, S. Y. et al. Cell 73 (7)1255-1266(1993)). Inhibition by these inhibitors illustrates very well the need for de novo transcription and protein synthesis, respectively, for the ‘execution’ of the hypersensitive response. Most measurable cellular responses associated with the hypersensitive response are suppressed effectively upon transcription inhibition, including cell death induction. Although the mechanism by which recognition of a pathogen avirulence protein by the plant is converted into an altered transcription pattern is not understood in much detail, it is likely that transcription factors mediate this effect. Since the hypersensitive response is a phenomenon found in almost all plant species, it is believed that at least the steps involving de novo transcription and protein synthesis are common between those plants and will be effected by tightly regulated transcription factors. It is believed that the transcription factor of the present invention can replace the endogenous plant transcription factors without being regulated. In this way, the presence or absence of the transcription factor of the invention acts as an on/off signal for the generation of an HR.

Transcription factors regulate gene expression by exerting their effects on a promoter. What most people would call ‘promoters’ consists of basically two different elements. The minimal promoter is the area where the basic transcription machinery, RNA polymerase and associated proteins bind, unwind the DNA and start transcription.

Flanking that minimal promoter, but in plants usually upstream from the minimal promoter (measured from the transcribed area), many different binding sites for transcription factors are found. This area is usually called ‘enhancer’, but also other descriptions, such as ‘silencer’ can be used (often based on the nature of the influence of the transcription factors).

This area may bind transcription factors, which have an effect on the ability of the basic transcription machinery to bind, unwind or initiate the transcription from the minimal promoter. Therefore they directly influence the transcription rate.

The activation or inactivation of the transcription rate by transcription factors may occur from some distance of the minimal promoter, but usually in plants, the most influential transcription factors work from within 1.5 kb upstream of the minimal promoter.

Transcription factors, and especially the transcription-activating ones appear to have a modular structure. They contain a DNA-binding domain, frequently characterized by a high incidence of basic amino acids. In addition, some contain dimerisation domains, which allow them to homo- or heterodimetize with other transcription factors. Examples of DNA-binding domains (sometimes linked to dimerisation domains) are bHLH, bZIP, bHLH-ZIP, helix-turn helix, POU and Zinc-fingers)

Most transcription factors also have a transcription activation domain which is usually separable from the DNA-binding and dimerisation domains. Transcription-activating domains are frequently characterized by glutamine-rich stretches, proline-containing areas, acidic domains or isoleucine-containing regions. Some transcription activation domains, however, are not characterized by any of the descriptions given above.

Many transcription factors, but not all, appear to have some level of regulation to their activity. Some are sequestered outside the nucleus by inhibiting proteins. These complexes can disrupt after signal transduction, leading to migration of the transcription factor to the nucleus, binding of DNA and activation of transcription from nearby promoters. Others need to complex with small molecules, such as hormones, to fold into a form that allows DNA-binding and transcription activation. Yet others are able to bind the DNA, but have transcription activation domains that need to be post-translationally modified to fully exert their function. No doubt, several more activation mechanisms exist.

Transcription activation domains can be identified through deletion studies on the transcription factor itself, but also by their ability to activate transcription when linked to heterologous DNA-binding domains (from other transcription factors).

In this set up, usually a reporter gene is used, where upstream of the minimal promoter, DNA-binding sites are introduced which can be bound by the DNA-binding domains mentioned before.

Most DNA-binding domains by themselves are unable to stimulate transcription from the nearby minimal promoter even when bound to their DNA-binding sites. By linking these DNA-binding domains to with parts of the transcription factor studied, one can easily identify the regions that mediate transcription activation, by analysing the reporter gene expression rate.

A review of transcription factors present in plants can be found in Meshi, T. and Iwabuchi, M. (1995) Plant Cell Physiology 36 (8), 1405-1420.

Although the invention is illustrated in detail for tomato and tobacco plants, it should be understood that any plant species in which transcription of genes involved in the execution of the hypersensitive response can be regulated by the protein of the invention may be provided with one or more plant expressible gene constructs, which when expressed are capable of inducing a HR-response. The invention can even be practiced in plant species that are presently not amenable for transformation, as the amenability of such species is just a matter of time and because transformation as such is of no relevance for the principles underlying the invention. Hence, plants for the purpose of this description shall include angiosperms as well as gymnosperms, monocotyledonous as well as dicotyledonous plants, be they for feed, food or industrial processing purposes; included are plants used for any agricultural or horticultural purpose including forestry and flower culture, as well as home gardening or indoor gardening, or other decorative purposes.

In order to provide a quick and simple test if a new plant species indeed can yield a hypersensitive response upon presentation of the Cladosporium fulvum elicitor the person skilled in the art can perform a rapid transient expression test known under the name of ATTA (Agrobacterium tumefaciens Transient expression Assay). In this assay (of which a detailed description can be found in Van den Ackerveken, G., et al., (Cell 87, 1307-1316, 1996) the nucleotide sequence coding for the Cladosporium fulvum elicitor is placed under control of a plant constitutive promoter and introduced into an Agrobacterium strain which is also used in protocols for stable transformation. After incubation of the bacteria with acetosyringon or any other phenolic compound which is known to enhance Agrobacterium T-DNA transfer, 1 ml of the Agrobacterium culture is infiltrated into an in situ plant by injection after which the plants are placed in a greenhouse. After 2-5 days the leaves can be scored for occurrence of BR symptoms.

Alternatively, for such a simple test. the Cladosporium fulvum-derived elicitor is placed under control of a plant constitutive promoter and introduced directly into plants or plant cells, using direct DNA delivery techniques, such as ‘biolistics’ or PEG-mediated transformation. A further, rapid way of testing the functionality is to use the PVX-derived expression system as described in the experimental section.

### Overexpression of Proteins

Proteins of the invention, also denominated Cladosporim fulvum elicitor, include all proteins comprising the amino acid sequence of SEQ ID NO:1 and muteins thereof.

The word protein means a sequence of amino acids connected trough peptide bonds. Polypeptides or peptides are also considered to be proteins. Muteins of the protein of the invention are proteins that are obtained from the proteins depicted in the sequence listing by replacing, adding and/or deleting one or more amino acids, while still retaining their HR-response inducing activity. Such muteins can readily be made by protein engineering in vivo, e.g. by changing the open reading frame capable of encoding the protein so that the amino acid sequence is thereby affected. As long as the changes in the amino acid sequences do not altogether abolish the activity of the protein such muteins are embraced in the present invention. Further, it should be understood that muteins should be derivable from the proteins depicted in the sequence listing while retaining biological activity, i.e. all, or a great part of the intermediates between the mutein and the protein depicted in the sequence listing should have HR-response inducing activity. A great part would mean 30% or more of the intermediates, preferably 40% of more, more preferably 50% or more, more preferably 60% or more, more preferably 70% or more, more preferably 80% or more, more preferably 90% or more, more preferably 95% or more, more preferably 99% or more.

Preferred muteins are muteins in which the first 90 amino acids as shown in SEQ ID NO:2 are deleted (and where the expressed protein starts with the Met-residue on amino acid position 91 of SEQ ID NO:2). Other preferred muteins are muteins with a mutation in the DNA-binding or leucine zipper domain, such as a mutein in which the Asn-residue on amino acid position 207 is replaced with an Ala-residue or a mutein in which the Leu-residue on amino acid positions 225, 239 or 253 is replaced with an Ala-residue. Also preferred are muteins with combinations of the above-mentioned deletions or mutations.

The protein of the invention comprises a distinct DNA-binding domain (amino acids 202-221) and a leucine zipper domain (amino acids 222-259). It is believed that, as indicated in the experimental section below, that conservation of these regions is essential for the function of the protein, although some variation is allowable. However, the other parts of the protein are less important for the function and may be more susceptible to change. Thus, also part of the invention are proteins in which the DNA-binding domain and the leucine domain are 80% or more identical with the domains of SEQ ID NO:2 and in which the other part of the sequence is 60% or more identical with the sequence of SEQ ID NO:2. For calculation of precentage identity the BLAST algorithm can be used (Altschul et al., 1997 Nucl. Acids Res. 25:3389-3402) using default parameters or, alternatively, the GAP algorithm (Needleman and Wunsch, 1970 J. Mol. Biol. 48:443-453), using default parameters, which both are included in the Wisconsin Genetics Software Package, Genetics Computer Group (GCG), 575 Science Dr., Madison, Wis., USA. BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, 1993 Comput. Chem. 17:149-163) and XNU (Clayerie and States, 1993 Comput. Chem. 17:191-201) low-complexity filters can be employed alone or in combination.

As used herein, ‘sequence identity’ or ‘identity’ in the context of two protein sequences (or nucleotide sequences) includes reference to the residues in the two sequences which are the same when aligned for maximum correspondence over a specified comparison window. When percentage of sequence identity is used in reference to proteins it is recognised that residue positions which are not identical often differ by conservative amino acid substitutions, where amino acids are substituted for other amino acid residues with similar chemical properties (e.g. charge or hydrophobicity) and therefore do not change the functional properties of the molecule. Where sequences differ in conservative substitutions, the percentage sequence identity may be adjusted upwards to correct for the conservative nature of the substitutions. Sequences, which differ by such conservative substitutions are said to have ‘sequence similarity’ or ‘similarity’. Means for making these adjustments are well known to persons skilled in the art. Typically this involves scoring a conservative substitution as a partial rather than a full mismatch, thereby increasing the percentage sequence identity. Thus, for example, where an identical amino acid is given a score of 1 and a non-conservative substitution is give a score of zero, a conservative substitution is given a score between 0 and 1. The scoring of conservative substitutions is calculated, e.g. according to the algorithm of Meyers and Miller (Computer Applic. Biol. Sci. 4:11-17, 1988).

As used herein, percentage of sequence identity means the value determined by comparing two optimally aligned sequences over a comparison window, wherein the protion of the amino acid sequence or nucleotide sequence in the comparison window may comprise additions or deletions (i.e., gaps) as compared to the reference sequence for optimal alignment of the two sequences. The percentage is calculated by determining the number of positions at which the identical amino acid or nucleic acid base residue occurs in both sequences to yield the number of matched positions, dividing the number of matched positions by the total number of positions in the window of comparison and multiplying the result by 100 to yield the percentage of sequence identity.

The amino terminal domain of about 90 residues of SEQ ID NO:2 is very rich in glutamines. This domain or a portion of this domain may be involved in activation of transcription by binding to the TFIID complex. This stretch of 90 amino acids contains 15 glutamines (17%) but the percentage of glutamines in residues 32 to 73 consists of 15 glutamines (36%). Glutamine rich domains are often components of proteins involved in transcription and are found in practically all eukaryotes (Escher D., et al., 2000, Cell Biol. 20(8):2774-2782; Schwechheimer C., et al., 1998, Plant Mol. Biol. 36(2):195-204). Examples of such glutamine rich transcription factors are the human transcription factor Sp1 (20% glutamines in a stretch of 112 residues), the B-cell derived trabscrption factor OCT-2A (26% in 63 residues) and the TAT box binding protien (44% in 78 residues) (Gerber H. P., et al., 1994, Science 263:808-811). Poly-glutamine stretches are also capable of activating transcription when fused to the DNA binding domain of GLA4 in human and plant cells (Gerber et al., 1994; Schwechheimer et al. 1998). Gugneja S., et al., (1996, Mol. Cell Biol. 16(10):5708-5716) have shown that even a glutamine containing stretch of 17 glutamines of 176 residues (content <10%) is capable of activating transcription.

Also included in the invention are chimeric transcription factors which have the DNA binding domain (amino acids 202-221) of SEQ ID NO:2 or muteins thereof, optionally a domain which is essential for dimerisation, such as the leucine zipper domain (amino acids 222-259) of SEQ ID NO:2, and a transcription activating domain. As illustrated before, such transcription activating domains may be characterized by glutamine-rich stretches, proline-containing areas, acidic domains or isoleucine-containing regions. The transcription activating domain preferably is expressly suited to stimulate the activity of plant promoters.

Also part of the invention are the nucleotide sequences coding for the protein of the invention and the above-described muteins. A preferred nucleotide sequence is the sequence as depicted in SEQ ID NO:1 from nucleotide 2 (atg start) to nucleotide 782 (tag stop) or conservatively modified or polymorphic variants thereof. Those of skill in the art will recognise that the degeneracy of the genetic code allows for a plurality of nucleotide sequences to encode for the identical amino acid sequence. Such “silent variations” can be used, for example, to selectively hybridise and detect allelic variants of the nucleotide sequences of the present invention. Other variations may be engineered to allow for codon optimisation, whereby a codon may be replaced with another codon encoding the same amino acid to adapt to the codon usage of the host organism.

The present invention provides a chimeric DNA sequence which comprises a pathogen inducible promoter which regulates the expression of the Cladosporium fulvum elicitor which is capable of eliciting a hypersensitive response. The expression chimeric DNA sequence shall mean to comprise any DNA sequence which comprises DNA sequences not naturally found in nature. The open reading frame may be incorporated in the plant genome wherein it is not naturally found, or in a replicon or vector where it is not naturally found, such as a bacterial plasmid or a viral vector. Chimeric DNA shall not be limited to DNA molecules which are replicable in a host, but shall also mean to comprise DNA capable of being ligated into a replicon, for instance by virtue of specific adaptor sequences, physically linked to the nucleotide sequence according to the invention.

The open reading frame coding for the Cladosporium fulvum elicitor may be derived from a genomic library. In this latter it may contain one or more introns separating the exons making up the open reading frame that encodes the protein. The open reading frame may also be encoded by one uninterrupted exon, or by a cDNA to the mRNA encoding the Cladosporium fulvum elicitor. Open reading frames according to the invention also comprise those in which one or more introns have been artificially removed or added. Each of these variants is embraced by the present invention.

Pathogen inducible promoters are known in the art and are responsive to a large number of pathogens and to aspecific elicitors produced by these pathogens. Examples of such pathogen inducible promoters are: the prp1 promoter (Martini, N., et al., Mol. Gen. Genet. 236, 179-186, 1993), the Fisl promoter (WO 96/34949), the Bet v 1 promoter (Swoboda, I., et al., Plant, Cell and Env. 18, 865-874, 1995), the Vst1 promoter (Fischer, R., Dissertation, Univ. of Hohenheim, 1994; Schubert, R., et al. Plant Mol. Biol. 34, 417-426, 1997), the sesquiterpene cyclase promoter (Yin, S., et al., Plant Physiol. 115, 437-451, 1997), the MS59 promoter (WO 99/50428), the ICS promoter (WO 99/50423) and the gstA1 promoter (Mauch, F. and Dudler, R., Plant Physiol. 102, 1193-1201, 1993). Several other promoters are known in the art and can be used to drive expression of the nucleotide sequences of this invention.

In eukaryotic cells, an expression cassette usually further comprises a transcriptional termination region located downstream of the open reading frame, allowing transcription to terminate and polyadenylation of the primary transcript to occur. In addition, the codon usage may be adapted to accepted codon usage of the host of choice. The principles governing the expression of a chimeric DNA construct in a chosen host cell are commonly understood by those of ordinary skill in the art and the construction of expressible chimeric DNA constructs is now routine for any sort of host cell, be it prokaryotic or eukaryotic.

In order for the open reading frame to be maintained in a host cell it will usually be provided in the form of a replicon comprising said open reading frame according to the invention linked to DNA which is recognised and replicated by the chosen host cell. Accordingly, the selection of the replicon is determined largely by the host cell of choice. Such principles as govern the selection of suitable replicons for a particular chosen host are well within the realm of the ordinary skilled person in the art.

A special type of replicon is one capable of transferring itself, or a part thereof, to another host cell, such as a plant cell, thereby co-transferring the open reading frame according to the invention to said plant cell. Replicons with such capability are herein referred to as vectors. An example of such vector is a Ti-plasmid vector which, when present in a suitable host, such as Agrobacterium tumefaciens, is capable of transferring part of itself, the so-called T-region, to a plant cell. Different types of Ti-plasmid vectors (vide: EP 0 116 718 B1) are now routinely being used to transfer chimeric DNA sequences into plant cells, or protoplasts, from which new plants may be generated which stably incorporate said chimeric DNA in their genomes. A particularly preferred form of Ti-plasmid vectors are the so-called binary vectors as claimed in (EP 0 120 516 B1 and U.S. Pat. No. 4,940,838). Other suitable vectors, which may be used to introduce DNA according to the invention into a plant host, may be selected from the viral vectors, e.g. non-integrative plant viral vectors, such as derivable from the double stranded plant viruses (e.g. CaMV) and single stranded viruses, gemini viruses and the like. The use of such vectors may be advantageous, particularly when it is difficult to stably transform the plant host. Such may be the case with woody species, especially trees and vines.

The expression “host cells incorporating a chimeric DNA sequence according to the invention in their genome” shall mean to comprise cells, as well as multicellular organisms comprising such cells, or essentially consisting of such cells, which stably incorporate said chimeric DNA into their genome thereby maintaining the chimeric DNA, and preferably transmitting a copy of such chimeric DNA to progeny cells, be it through mitosis or meiosis. According to a preferred embodiment of the invention plants are provided, which essentially consist of cells which incorporate one or more copies of said chimeric DNA into their genome, and which are capable of transmitting a copy or copies to their progeny, preferably in a Mendelian fashion. By virtue of the transcription and translation of the chimeric DNA according to the invention in some or all of the plant's cells, those cells that are capable of producing the Cladosporium elicitor upon infection with a pathogen will show enhanced resistance to fungal infections.

Transformation of plant species is now routine for an impressive number of plant species, including both the Dicotyledoizeae as well as the Monocotyledoneae. In principle any transformation method may be used to introduce chimeric DNA according to the invention into a suitable ancestor cell, as long as the cells are capable of being regenerated into whole plants. Methods may suitably be selected from the calcium/polyethylene glycol method for protoplasts (Krens, F. A. et al., 1982, Nature 296, 72-74; Negrutiu I. et al, June 1987, Plant Mol. Biol. 8, 363-373), electroporation of protoplasts (Shillito R. D. et al., 1985 Bio/Technol. 3 1099-1102), microinjection into plant material (Crossway A. et al., 1986, Mol. Gen. Genet. 202, 179-185), (DNA or RNA-coated) particle bombardment of various plant material (Klein T. M. et al., 1987, Nature 327, 70), infection with (non-integrative) viruses and the like. A preferred method according to the invention comprises Agrobacterium-mediated DNA transfer. Especially preferred is the use of the so-called binary vector technology as disclosed in EP A 120 516 and U.S. Pat. No. 4,940,838. Tomato transformation is preferably done essentially as described by Van Roekel et al. (Van Roekel, J. S. C., Damm, B., Melchers, L. S., Hoekema, A. (1993). Factors influencing transformation frequency of tomato (Lycopersicon esculeiztuiii). Plant Cell Reports, 12, 644-647). Potato transformation is preferably done essentially as described by Hoekema et al. (Hoekema, A., Huisman, M. J., Molendijk, L., van den Elzen, P. J. M., and Cornelissen, B. J. C. (1989). The genetic engineering of two commercial potato cultivars for resistance to potato virus X. Bio/Technology 7, 273-278). Generally, after transformation plant cells or cell groupings are selected for the presence of one or more markers which are encoded by plant expressible genes co-transferred with the nucleic acid sequence according to the invention, whereafter the transformed material is regenerated into a whole plant.

Although considered somewhat more recalcitrant towards genetic transformation, monocotyledonous plants are amenable to transformation and fertile transgenic plants can be regenerated from transformed cells or embryos, or other plant material. Presently, preferred methods for transformation of monocots are microprojectile bombardment of embryos, explants or suspension cells, and direct DNA uptake or electroporation (Shimamoto, et al, 1989, Nature 338, 274-276). Transgenic maize plants have been obtained by introducing the Streptomyces hygroscopicus bar-gene, which encodes phosphinothricin acetyltransferase (an enzyme which inactivates the herbicide phosphinothricin), into embryogenic cells of a maize suspension culture by microprojectile bombardment (Gordon-Kamm, 1990, Plant Cell, 2, 603-618). The introduction of genetic material into aleurone protoplasts of other monocot crops such as wheat and barley has been reported (Lee, 1989, Plant Mol. Biol, 13, 21-30). Wheat plants have been regenerated from embryogenic suspension culture by selecting only the aged compact and nodular embryogenic callus tissues for the establishment of the embryogenic suspension cultures (Vasil, 1990 Bio/Technol. 8, 429-434). The combination with transformation systems for these crops enables the application of the present invention to monocots.

Monocotyledonous plants, including commercially important crops such as rice and corn are also amenable to DNA transfer by Agrobacteriltn strains (vide WO 94/00977; EP 0 159 418 B1; Gould J, Michael D, Hasegawa O, Ulian E C, Peterson G, Smith R H, (1991) Plant. Physiol. 95, 426-434; Y. Hiei et al., (1994) The Plant J. 6, 271-282).

Following DNA transfer and regeneration, putatively transformed plants may be evaluated, for instance using Southern analysis, for the presence of the chimeric DNA according to the invention, copy number and/or genomic organization. After the initial analysis, which is optional, transformed plants showing the desired copy number and expression level of the newly introduced chimeric DNA according to the invention may be tested for resistance levels against a pathogen.

Other evaluations may include the testing of pathogen resistance under field conditions, checking fertility, yield, and other characteristics. Such testing is now routinely performed by persons having ordinary skill in the art.

Following such evaluations, the transformed plants may be grown directly, but usually they may be used as parental lines in the breeding of new varieties or in the creation of hybrids and the like. These plants, including plant varieties, with improved resistance against pathogens may be grown in the field, in the greenhouse, or at home or elsewhere. Plants or edible pails thereof may be used for animal feed or human consumption, or may be processed for food, feed or other purposes in any form of agriculture or industry. Agriculture shall mean to include horticulture, arboriculture, flower culture, and the like. Industries which may benefit from plant material according to the invention include but are not limited to the pharmaceutical industry, the paper and pulp manufacturing industry, sugar manufacturing industry, feed and food industry, enzyme manufacturers and the like.

The advantages of the plants, or parts thereof, according to the invention are the decreased need for pesticide treatment, thus lowering costs of material, labour, and environmental pollution, or prolonging shelf-life of products (e.g. fruit, seed, and the like) of such plants. Plants for the purpose of this invention shall mean multicellular organisms capable of photosynthesis, and subject to some form of pathogen induced disease. They shall at least include angiosperms as well as gymnosperms, monocotyledonous as well as dicotyledonous plants.

### EXPERIMENTAL PART

Standard methods for the isolation, manipulation and amplification of DNA, as well as suitable vectors for replication of recombinant DNA, suitable bacterium strains, selection markers, media and the like are described for instance in Maniatis et al., molecular cloning: A Laboratory Manual 2nd. edition (1989) Cold Spring Harbor Laboratory Press; DNA Cloning: Volumes I and II (D.N. Glover ed. 1985); and in: From Genes To Clones (E.-L. Winnacker ed. 1987).

### Example 1

### Construction of a cDNA Library of Cladosporium Fulvum in a Binary PVX Vector and Storage of the Library

Various strains of C. fulvum were nutrient-starved by culturing them for 10 to 16 days in B5 medium at 22° C. (De Wit and Flach, 1979), without refreshing the media. Under such conditions the fungus expresses genes that are predominantly induced upon colonisation of tomato leaves (Coleman et al., 1997; Van den Ackerveken et al., 1994), allowing isolation of RNA from the fungus, without contaminating plant RNAs. RNA was extracted from the mycelium by the hot-phenol procedure (Extract-A-Plant RNA isolation kit, Clontech, U.S.A.) and expression of Avr and Ecp genes was examined by northern blotting of 3.5 μg of total RNA from various strains separated on a 1% agarose/glyoxal gel (Sambrook et al., 1989). The RNA was transferred to Hybond N⁺ membrane (Amersham, U.K.), hybridised at 65° C. (Church and Gilbert, 1984) with ³²P-labelled DNA probes (Life Technologies, U.K.), washed at high stringency (0.1 SSC/0.5% SDS, 65° C.) and subsequently X-ray films were exposed to the blots. Probes for Avr4, Av/9, Ecp1, Ecp2, Ecp4 and Ecp5 were generated by PCR amplification of the cDNA inserts present in cloning vectors (Joosten et al., 1994; Laugé, 1999; Van den Ackerveken et al., 1993).

A race 5 strain was selected that, during nutrient starvation, showed relatively high expression of the various Avr- and Ecp-genes. From this strain poly(A)⁺ RNA was purified from total RNA using oligotex microbeads (Qiagen, U.S.A.). The Smart cDNA kit (Clontech, U.S.A.) was employed to construct cDNA with asymmetric SfiI sites, using a primer extension method, followed by a size separation step. Full-length cDNAs, that were larger than 250 base pairs, were digested with SfI and directionally cloned into a SfiI-digested, dephoshorylated, binary pSfinx vector. The pSfinx binary vector contains on its T-DNA a modified full length PVX genomic sequence, under control of the 35S CaMV promoter that drives plant expression of the viral genome. It contains a duplicated coat protein promoter that allows insertion of DNA sequences for overexpression. Briefly, pSfinx was derived from pGr106 (kindly provided by Dr. D. Baulcombe, Sainsbury Laboratory, Norwich, U.K.), by inserting four additional restriction sites (5′-SfiI/SmaI/EcoRV/SfiI-3′) between the ClaI and AscI sites present at the poly-linker downstream of the duplicated PVX coat protein promoter.

The ligation mixture was subsequently transformed to electro-competent Agrobacterium tumefaciens strain Mog101 (Hood et al., 1993), containing the helper plasmid pIC-SArep Jones et al., 1992), using a modification of the procedure described by Mersereau et al., 1990. In brief, electro-competent cells of Mog101 were obtained by growing them in TB (12 g/l tryptone, 24 g/l yeast extract, 0.4% glycerol (v/v), 0.017 M KH₂PO₄ and 0.072 M K₂HPO₄, pH), to an optical density at 660 nm of 1, followed by washing four times with distilled water and finally resuspending in 0.005 volumes of 10% (v/v) glycerol in water. The cDNA, ligated in pSfinx, was added to 40 μl of competent A. tumefaciens cells (in a concentration generally resulting in ca. 300 colonies per plate) followed by electro-transformation with a Gene Pulser (Biorad, U.S.A.). After recovery for three hours at 28° C. in 1 ml LB-mannitol (10 g/l tryptone, 5 g/l yeast extract, 2.5 g/l NaCl and 10 g/l mannitol), cells were plated on LB-mannitol agar, supplemented with 100 μg kanamycin and 20 μg rifampicin per ml and incubated at 28° C. for 2 days. Colonies were transferred to 96-wells micro-titer plates (Greiner, Germany) containing 100 μl TB per well, using a Flexys robotic workstation (Genomic Solutions, U.K.). Cells were grown for 2 days at 28° C. and glycerol was added to a final concentration of 30% before storing plates at −80° C.

### Example 2

### Functional Screening of the Library

For functional screening of the library on plants, the A. tumefaciens cultures were transferred from the 96-wells micro-plates to LB-mannitol agar plates, supplemented with antibiotics and incubated for 2 days at 28° C. With a toothpick, individual colonies were inoculated onto five-week-old tomato plants carrying either resistance gene Cf-4 (MM-Cf4) or Cf-9 (MM-Cf9) against C. fulvum, by piercing the leaves on both sides of the mid vein. In this way, 96 colonies were inoculated onto one tomato plant, with 8 colonies in duplicate on each of 12 leaflets. Putative positive clones were re-screened on the same tomato genotypes and on tobacco species Nicotiana clevelandii. For functional screening of the library on tobacco (N. tabacum var. Samsun NN), up to 5 expanded leaves per plant were inoculated with 96 colonies per leaf. Colonies were transferred simultaneously, using a 96-needle colony transfer device. Leaves were scored 11 to 20 days after inoculation for the presence of local HR, visible as a necrotic and/or chlorotic sector flanking the primary inoculation site, and for systemic HR.

To test whether this approach is feasible for functional screening of a cDNA library of C. fulvum, iii planta expression of wild type Avr4 and Avr9 cDNAs using binary vectors either containing (pSfinx) or lacking the PVX component (pAvr), were compared. In pSfinx the cDNAs are inserted downstream of the duplicated PVX coat protein promoter, whereas in pAvr the Avr-cDNAs are present downstream of the constitutive 35S promoter (Van der Hoorn et al., 2000). The resulting plasmids were transformed to A. tumefaciens, Mog101, and the four recombinant strains were toothpick-inoculated onto MM-Cf4 and MM-Cf9 plants. Only A. tumefaciens colonies containing the pSfinx constructs induce a visible HR when inoculated onto plants carrying the matching resistance gene. A. tumeacienzs containing pAvr does not induce an HR visible by the naked eye, indicating that the PVX component is essential for ensuring expression of sufficient amounts of elicitor in the plant and spreading of the lesion.

In earlier reports on in planta expression of Avr4 and Avr9, the fungal sequence encoding the signal peptide for extracellular targeting of the AVRs, was replaced by the sequence of the PR-1a signal sequence of tobacco (Hammond Kosack et al., 1994; Hammond Kosack et al., 1995, Honée et al., 1998, Joosten et al., 1997). Here we expressed Avr4 and Avr9 cDNAs containing the sequence encoding the native signal peptide. As clear genotype-specific necrosis was observed, correct targeting of the encoded proteins occurs in tomato, indicating that the native fungal signal sequence also function in planta.

To determine whether this binary PVX expression system is also functional in other hosts for A. tumefaciens and PVX, the bacteria carrying the Avr genes in the pSfinx vector were inoculated onto transgenic Nicotiana tabacum var. SR1, expressing either Cf4 or Cf-9 (Romeis et al., 1999; Takken et al., 1999). Clear necrosis developed when a matching Avr-Cf gene pair was present. In tobacco, necrosis remained confined to the tissue surrounding the wound site, whereas in tomato the lesions eventually spread systemically, resulting in death of the plant (results not shown).

Thus, A. tumefaciens-mediated delivery of a binary vector containing a CDNA of interest, inserted into PVX, is an efficient tool to express cDNAs encoding Avr4 and Avr9 of C. fulvum in both tomato and tobacco. These positive results-prompted us to use this system for high-throughput, functional screening of a cDNA library of C. fulvum grown in vitro, to identify cDNAs encoding ‘elicitors’ that specifically induce HR on particular genotypes of tomato and tobacco.

Functional Screening of the cDNA Library on Tomato and Recovery of HR Inducing Clones

Poly(A)+ RNA was isolated from strain 5a of C. fulvum, cDNA was synthesised, ligated into pSfinx and subsequently transformed to A. tumefaciens. Individual colonies were picked and a library consisting of 9,600 A. tumefaciens colonies, each containing the pSfinx vector with a cDNA insert, was stored. Analysis of 50 randomly selected clones, revealed that nearly all contained an insert varying in size between 250 to over 3500 base-pairs (bp), in the proper orientation (results not shown). The library was screened by toothpick-inoculation of each individual A. tumefaciens colony onto leaves of MM-Cf4 and MM-Cf9 plants. Between 11 to 20 days after inoculation, leaves were examined for development of necrosis or chlorosis around the inoculation site. Putative positive colonies were re-inoculated both onto tomato and Nicotiatia clevelandii, to determine the specificity of HR-inducing activity.

The screening eventually resulted in the identification of four different A. tumefaciens colonies that repeatedly gave HR on tomato (Table 1). Three of these colonies also induced HR on N. clevelandii. Colony 72-11F only induced HR on NM-Cf4 plants, whereas colony 84-5C gave HR on both MM-Cf4 and MM-Cf9 plants. Probably colonies 89-10A and 43-7G were missed in the first cultivar-specific screen on MM-Cf9 and MM-Cf4, respectively, as they induce non-cultivar specific HR. The three colonies that were found to be positive both on tomato and tobacco (43-7G, 84-5C and 89-10A), induce necrosis one to two days earlier than colonies giving a cultivarspecific HR

To identify the nature of the cDNAs of which functional expression induces HR, the clones present in the positive A. tumefaciens colonies were isolated and sequenced. The MM-Cf4-specific clone (72-11F) contains an open reading fiame (ORF) of 408 bp, a 5′untranslated region (UTR) of 55 bp and a 3′ UTR of 166 bp. The sequence was identical to the sequence published for the Avr4 MRNA encoding the AVR4 elicitor (Joosten et al., 1994).

The three cDNAs, of which functional expression induced lesions both on tomato and tobacco, are all about 830 bp in length. Sequencing revealed that these cDNAs all originate from the same gene. They are, however, clearly independent, as their 5′ UTRs and polyadenylation sites differ in all three cases. The CDNA contains an uninterrupted ORF of 510 bp, probably encoding a transcription factor of C. fulvum, as a Blast search (Altschul et al., 1997) revealed that the encoded protein contains a DNA-binding domain which has high homology to that of the family of B-Zip basic transcription factors. It is 42% homologous to the DNA-binding domain of the Drosophila FOS-related antigen (DFRA) transcription factor (Perkins et al. 1990), while it has 50% homology to the DNA-binding domain of the general control protein (GCN)-4 of Saccharomyces cerevisiae, a transcription factor involved in regulation of amino acid biosynthesis (Hinnebusch, 1984), while it had the highest homology with JUN of Avian sarcoma virus 17. All of these genes encode bZIP transcription factors, the highest homology was found in the basic DNA binding- and leucine zipper domain. Southern analysis of one of the three clones (43-7G) as a probe revealed that homologous sequences are present in C. fulvum races 0, 4 and 5 as a single copy gene.

To identify if homologous genes were present in plants, Southern blots of tomato, tobacco and Arabidopsis gDNA were probed with labelled 43-7G cDNA. No specific hybridisation signal was found using low stringent hybridisation and washing conditions (55° C., 3×SSC/0.1%SDS). This indicates that at the DNA level no highly homologous genes are present in these plant species. Plants do have bZfP proteins, though (see e.g. Schindler et al., 1992. EMBO J. 11:1261-73)

### Example 3

### Isolation and Sequencing of cDNA Clones of Which Functional Expression Causes HR

Colonies of A. tumefaciens that caused local and/or systemic HR after re-screening, were grown in TB supplemented with antibiotics. Subsequently plasmid DNA was isolated by alkaline lysis and transformed to electro-competent E. coli DH5α, according to standard procedures (Sambrook et al., 1989). Inserts were isolated either by PCR, using the primers OX10 (5′-CAATCACAGTGTTGGCTTGC-3′) (SEQ ID NO:3) and N31 (5′-GACCCTATGGGCTGTGTTG-3′) (SEQ ID NO:4) that flank the cDNA insert, or by digestion with ClaI and NotI. The cDNA inserts were sequenced with the Big Dye-terminator method (Perkin Elmer, U.S.A.) using either the OX10 or N31 primers.

Colony Hybridisation of the cDNA Library

For colony hybridisation, cultures of A. tumefaciens were transferred from the 96-wells micro-titer plates to LB-mannitol agar plates, supplemented with kanamycin and rifampicin. Colonies were grown for 2 days at 28° C. and transferred to Hybond N⁺ membranes (Amersham U.K.). The bacteria were subsequently lysed and the released DNA was fixed to the membranes using standard procedures (Sambrook et al., 1989), with the modification that the lysis step was prolonged to 15 minutes. After denaturing and neutralisation, the filters were hybridised with various probes similar to probing the northern blots.

### Example 4

Expression of 43-7G Induces Necrosis in Many Plant Species

Necrosis-inducing activity of 43-7G was examined by tooth-pick inoculation of Agrobacierium with pSfinx::43-7G of near-isogenic tomato (var. Moneymaker) lines Cf-2, Cf-4, Cf-5, Cf-9, Cf-veda and Cf-18, and 11 Nicotiana spp. Except for N. sylvestris all plants showed 43-7G specific necrosis (FIG. 1). Some plant only showed necrosis at the inoculation site (N. rustica, N. tabacum, N. paniculata and N. solanifolia), while others showed systemic necrosis in both the inoculated and the higher uninoculated leaves (all tomato varieties, N. benthamiana, N. clevelandii, N. glutinosa, N. cordifolia and N. langsdorfii). The severity of necrotic systems varied in these species possibly because of differences in expression levels of 43-7G caused by more or less efficient replication of the virus in this host. N. sylvestris, for example, did not show HR-like symptoms using the PVX system, but showed necrosis upon ATTA infiltration (Van der Hoorn et al., 2000 MPMI 13:438-446) of a binary vector expressing 43-7G in Agrobacterium.

### Example 5

### Identification of Essential Domains

The largest ORF of SEQ ID NO:1 starts at the ATG (at position 3 in the sequence listing, ATG3) in the polylinker and continues till the stop codon (TAG) at position 780. In the same reading frame a second ORF is present (ATG273-TAG780), which might encode the protein that is actually functional in C. fitlvum. Between these two start codons three additional ATGs are present (ATGIO2, 155 and 262) which are not in the same reading frame. To identify which ATG is essential for the HR-inducing activity two deletion mutants were made: 43-7G*79 (only removing the ATG in the poly-linker) and 43-7G*265 (removing all ATG's except the one of the second ORF in the same reading frame). These were cloned into pSfinx and used for Agrobacterium mediated inoculation. To our surprise, these constructs showed a different spectrum of necrosis-inducing activity in various plant species. As before, clear necrosis was observed in tomato, N. tabacum and N. clevelandii in control, non-mutated 43-7G inoculated plants. No necrosis was observed with these plant species when inoculated with 43-7G*79, but when inoculated with 43-7G*265 N. tabacumii showed necrosis, while tomato and N. clevelaindii did not. (FIG. 2). The ability of the latter construct to induce necrosis was investigated further by inoculation of other tobacco species using sap containing infectious virus particles. N. benthamiana and N. langsdorfi showed severe necrosis, while some milder symptoms were seen on N. tabacum, N. glutintosa and N. solanifolia. No necrotic symptoms were observed in inoculated N. paniculata and N. sylvestris leaves.

Further six mutants of 43-7G were made to study the effect of disturbance of the DNA binding and the leucine zipper domain. The DNA binding domain stretches from amino acid 202 (Arg) to amino acid 221 (Arg), while the leucine zipper domain stretches from amino acid 222 (Ala) amino acid 259 (Lys), as indicated in SEQ ID NO:2. Two point mutations in codons for conserved amino acids were introduced in the DNA-binding domain, one replacing the codon for amino acid Asn for a codon for amino acid Ala at position 207, the other replacing two codons coding for Arg with codons coding for Ala at positions 215 and 217. Further a mutant was made with a small deletion, missing amino acids 202-207 (Arg-Lys-Arg-Gln-Arg-Asn). For the leucine zipper domain three mutants were made with point mutations changing Leu to Ala at positions 225, 239 and 253, respectively. All six constructs (and a full length 43-7G construct as control) were inserted into a binary vector under control of a 35S promoter, and examined for HR-inducing activity by ATTA on N. langsdorfii. The ATTAs were repeated 3 times, results are shown in Table 2. The results show clearly that the DNA binding domain, which is characterised by the basic amino acids, is extremely important for induction of HR, and that also the conserved leucine residues in the leucine zipper motif are important to determine its HR-inducing activity. Both elements are known to be essential for DNA binding, the basic region interacts directly with the DNA helix, and the leucine zipper motif serves as a dimerisation domain for this class of proteins.

TABLE 1
Number of HR-inducing colonies after the first functional screen and
position of positive colonies remaining after the second screen
of a cDNA library of strain 5a of C. fulvum,
on MM-Cf4 and MM-Cf9 tomato.

| Near- | | | |
|---|---|---|---|
| isogenic | HR-inducing clones | Position HR-inducing | |
| line | 1^{st} screen^{a)} | clones, 2^{nd} screen | Specificity^{b)} |
| MM-Cf4 | 61 | 72-11F | MM-Cf4 |
| | | | specific |
| | | 84-5C | Non-specific |
| | | 89-10A | Non-specific |
| MM-Cf9 | 34 | 43-7G | Non-specific |
| | | 84-5C | Non-specific |

^{a}The library was screened in two rounds; colonies that gave HR-like symptoms in the first screening round of 9,600 colonies, were re-screened.
^{b}Colonies, that induced a clear HR in the second round, were also screened on N. clevelandii and both tomato genotypes for specificity of HR-inducing activity. ‘Non-specific’ indicates that the colony induces HR on all near-isogenic lines of tomato and on N. clevelandii.

TABLE 2
ATTA infiltrations of binaty vectors containing various
constructs, on N. langsdorfii. HR-inducing activity of the
constructs is indicated as percentage of necrotic area of the
total infiltrated region.

| | HR-inducing |
|---|---|
| Construct | activity (%) |
| 43-7G full length cDNA | 100 |
| Mutant 1 DNA binding domain (N207→A) | 100 |
| Mutant 2 DNA binding domain (R215→A, R217→A) | 10-25 |
| Mutant 3 DNA binding domain (deletion R202→N207) | 0-5 |
| Mutant 1 Leucine zipper (L225→A) | 100 |
| Mutant 2 Leucine zipper (L239→A) | 60-80 |
| Mutant 3 Leucine zipper (L253→A) | 60-80 |

### REFERENCES

Altschul, S. F., Madden, T. L., Schaiffer, A. A., Zhang, J., Miller, W. and D. J., L. (1997) Gapped Blast and PSI-Blast: a new generation of protein database search programs. Nucleic Acid Research 25, 3389-3402.

Baker, B., Zambryski, P., Staskawicz, B. and Dinesh-Kumar, S. P. (1997) Signaling in Plant Microbe interactions. Science 276, 726-733.

Balhadere, P. V., Foster, A. J. and Talbot, N. J. (1999) Identification of pathogenicity mutants of the rice blast fungus Magnaporthe grisea by insertional mutagenesis. Mol. Plant Microbe Interact. 12, 129-142.

Bent, A. F. (1996) Plant disease resistance genes—function meets structure. Plant Cell 8, 1757-1771.

Bonas, U. and Van den Ackerveken, G. (1999) Gene-for-gene interactions: bacterial avirulence proteins specify plant disease resistance. Curr. Opin. Microbiol. 2, 94-98.

Chapman, S., Kavanagh, T. and Baulcombe, D. (0.1992) Potato virus X as a vector for gene expression in plants. Plant J. 2, 549-557.

Church, G. M. and Gilbert, W (1984) Genomic sequencing. Proc. Natl. Acad. Sci. USA 81, 1991-1995.

Coleman, M., Henricot, B., Arnau, J. and Oliver, R. P. (1997) Starvation induced genes of the tomato pathogen Cladosporum fulvum are also induced during growth in planta. Mol. Plant Microbe Interact. 10, 1106-1109.

Cui, Y.,N., B., Jackman, I., Bi, Y.-M. and Rothstein, S. J. (1999) Structural and transcriptional comparative analysis of the S locus regions in two self-incompatible Brassica napus lines. Plant Cell 11, 2217-2231.

De Wit, P. J. G. M. and Flach, W. (1979) Differential accumulation of phytoalexins in tomato leaves, but not in fruits, after inoculation with virulent and avirulent races of Cladosporium fulvum. Physiological Plant Pathology 15, 257-267.

Flor, H. H. (1942) Inheritance of pathogenicity in Melampsora lini. Phytopath. 32, 653-669.

Hammond Kosak, K. E., Harrison, K. and Jones, J. D. G. (1994) Developmentally regulated cell death on expression of the fungal avirulence gene Avr9 in tomato seedlings carrying the disease resistance gene CJ9. Proc.Natl. Acad. Sci. USA 91, 10445-10449.

Hammond Kosack, K. E. staskawicz, B. J., Jones, J. D. g. and Baulcombe, D. C. (1995) Functional expression of a fungal avirulence gene from a modified potato virus x genome. Mol. Plant microbe Interact. 8, 181-185.

Hinnebusch, A. G. (1984) Evidence for translational regulation of the activator of general amino acid control in yeast. Proc. Natl. Acad. Sci US 81, 6442-6446.

Honée, G., Buitink, J., Thorsten, J., De Kloe, J., Sijbolts, F., Apotheker, M., Weide, R., Sijen, T., Stuiver, M. and De Wit, P. J. G. M. (1998) Induction of defense related responses in CJ9 tomato cells by the AVR9 elicitor peptide of Cladosporium fulvum is developmentally regulated. Plant Phys. 117, 809-820.

Hood, E. E., Gelvin, S. B., Melchers, L. S. and Hoekema, A. (1993) New Agrobacterium helper plasmids for gene transfer to plants. Transgenic Res. 2, 208-218.

Jones, J. D. G. Shlumukov, L., Carland, F. J., Scofield, S., Bishop, G. and Garrison, K. (1992) Effective vectors for transformation, expression of heterologous genes, and assaying transposon excision in transgenic plants. Transgene Res. 1, 285-297.

Joosten, M. H. A. J., Cozijnsen, T. J. and De Wit, P. J. G. M. (1994) Host resistance to a fungal tomato pathogen lost by a single base pair change in an avirulence gene. Nature 367, 384-386.

Joosten, M. H. A. J. and De Wit, P. J. G. M. (1999) The tomato Cladosporium fulvum interaction: a versatile experimental system to study plant pathogen interactions. Annu. Rev. Phytopathol. 37, 355-367.

Joosten, M. H. A. J., Vogelsang, R., Cozijnsen, T. J., Verberne, M. C. and De Wit, P. J. G. M. (1997) The biotrophic fungus Cladosporium fulvum circumvents Cf4 mediated resistance by producing unstable AVR4 elicitors. Plant Cell 9, 367-379.

Kamoun, S., honce, G., Weide, R., Lauge, R., Kooman-Gersmann, M. de Groot, K., Govers, F. and De Wit, P. J. G. M. (1999) The fungal gene Avr9 and the oomycete gene Infl confer avirulence to potato virus X on tobacco. Mol. Plant Microbe Interact. 12, 459-462.

Kang, S., Sweigard, J. A. and Valent, B. (1995) The PWL host specificity gene family in the blast fungus Magnaporthe grisea. Mol. Plant Microbe Interact. 8, 939-948.

Karrer, E. E., Beachy, R. N. and Holt, C. A. (1998) Cloning of tobacco genes that elicit the hypersensitive response. Plant Mol. Biol. 36, 681-690.

Knogge, W. (1996) Fungal infection of plants. Plant Cell 8, 1711-1722.

Laugé, R. (1999) in Extracellular proteins of the tomato pathogen Cladosporium fulvum: role in pathogenicity and avirulence, PhD thesis Phytopathology, Wageningen University, Wageningen, pp. 98, ISBN 90 5485 985 7.

Laugé, R. and De Wit, P. J. G. M. (1998) Fungal avirulence genes: structure and possible functions. Fungal Genetics and Biology 24, 285-297.

Laugé, R., Goodwin, P. H., Joosten, M. H. A. J. and De Wit, P. J. G. M. (submitted) Specific HR associated recognition of secreted proteins form Cladosporium fulvum occurs in both host and non host plants.

Laugé, R., Joosten, M. H. A. J., Haanstra, J. P. W., Goodwin, P. H., Lindhout, P. and De Wit, P. J. G. M. (1998) Successful search for a resistance gene in tomato targeted against a virulence factor of a fungal pathogen. Proc. Natl. Acad. Sci. USA 95, 9014-9018.

Laugé, R., Joosten, M. H. A. J., Van Den Ackerveken, G. F. J. M. Van Den Broek, H. W. J. and De Wit, P. J. G. M. (1997) The in planta produced extracellular protein ECP1 and ECP2 of Cladosporium fulvum are virulence factors. Mol. Plant Microbe Interact 10, 735-744.

MacFarlane, S. A. and Popovich, A. H. (2000) Efficient expression of foreign proteins in roots from tobravirus vectors. Virology 267.

Mersereau, M., Pazour, G. J. and Das, A. (1990) Efficient transformation of Agrobacterium tumefaciens by electroporation. Gene 90, 149-151.

Nürnberger, T. (1999) Signal perception in plant pathogen defense. Cell. Molec. Life. Sci. 55, 167-182.

Perkins, K. K., Admon, A., Patel, N. and Tjian, R. (1990) The Drosophila Fos related AP 1 protein is a developmentally regulated transcription factor. Genes Dev. 4, 822-834.

Romeis, T., Piedras, P., Zhang, S. Q., Klessig, D. F., Hirt, H. and Jones, J. D. G. (1999) Rapid Avr9 and Cf9 dependent activation of MAP kinases in tobacco cell cultures and leaves: Convergence of resistance gene, elicitor, wound, and salicylate responses. Plant Cell 11, 273-287.

Sambrook, J., Fritsch, E. F. and Maniatis, T. (1989) in: Molecular Cloning: A Laboratory Manual., Vol. 2nd (2nd, Ed.) Cold Spring Harbor Laboratory Press, NY.

Takken, F. L. W., Thomas, M., Joosten, M. H. A. J., Goistein, C., Westerink, N., Hille, J., Nijkamp, H. J. J., De Wit, P. J. G. M. and Jones, J. D. G. (1999) A second gene at the tomato Cf4 locus confers resistance to Cladosporiuni fiilvumn through recognition of a novel avirulence determinant. Plant J. 20, 279-288.

Tichelaar, E. C. (1984) Collection of isogenic tomato stocks. Rep. Tomato. Genet. Coop. 334, 55-57.

Valent, B. (1997) in: The lice blast fungus Magnaporthe grisea, pp. pp. 37-54 (G. C. Carroll and P. Tudzynski, E., Ed.) Springer Verlag, Berlin.

Van den Ackerveken, G. F. J. M., Dunn, R. M., Cozijnsen, A. J., Vossen, J. P., Van den Broek, H. W. and De Wit, P. J. G. M. (1994) Nitrogen limitation induces expression of the avirulence gene avr9 in the tomato pathogen Cladosporium fulvum. Mol. Gen. Genet. 243, 277-285.

Van den Ackerveken, G. F. J. M., Van Kan, J. A., Joosten, M. H. A. J., Muisers, J. M., Verbakel, H. M. and De Wit, P. J. G. M. (1993) Characterization of two putative pathogenicity genes of the fungal tomato pathogen Cladosporim fulvum. Mol. Plant Microbe Interact. 6, 210-215.

Van den Ackerveken, G. F. J. M., Van Kan, J. A. L. and De Wit, P. J. G. M. (1992) Molecular analysis of the avirulence gene Avr9 of the fungal tomato pathogen Cladosporium fulvum fully supports the gene for gene hypothesis. Plant J. 2, 359-366.

Van der Hoorn, R. A. L., Laurent, F., Roth, R. and De Wit, P. J. G. M. (2000) Agroinfiltration is a versatile tool that facilitates comparative analyses of Avr9/Cf9 and Avr4/Qf4 induced necrosis. Mol. Plant Microbe Interact. 13, 438-446.

Van Kan, J. A. L., Van den Ackerveken, G. F. J. M. and De Wit, P. J. G. M. (1991) Cloning and characterization of CDNA of avirulence gene Avr9 of the fungal pathogen Cladosporium fulvum, causal agent of tomato leaf mold. Mol. Plant Microbe Interact. 4, 52-59.

Waterhouse, P. M., Smith, N. A. and Wang, M. B. (1999) Virus resistance and gene silencing: killing the messenger. Trends. Pl. Sci. 4, 452-457.

1. Method for the induction of pathogen resistance in plants characterized by transforming a plant with a polynucleotide sequence comprising a pathogen inducible promoter which regulates the expression of a Cladosporium transcription factor protein comprising an amino acid sequence as depicted in SEQ ID NO: 2 or a mutein thereof which when expressed gives rise to a hypersensitive response in plants.

2. A protein obtainable from Cladosporium fulvum and capable of eliciting a HR response in plants characterised in that it comprises the amino acid sequence of SEQ ID NO: 2 or a mutein thereof.

3. A nucleotide sequence encoding for a protein according to claim 2.

4. A nucleotide sequence according to claim 3, characterised in that it comprises the nucleotide sequence from base pairs 2-785 of SEQ ID NO: 1.

5. A chimaeric nucleotide sequence comprising the nucleotide sequence of claim 3 or 4 under operational control of a pathogen inducible promoter.

6. A vector comprising the chimaeric nucleotide sequence of claim 5.

7. A host comprising a vector according to claim 6.

8. A host according to claim 7 characterised in that it is Agrobacterium.

9. Method for transforming a plant using a host according to claim 8.

10. Method according to claim 1 or 9 to make a plant resistant against plant pathogens.

11. Plant made resistant against plant pathogens according to the method of claim 1,9 or 10.


SEQUENCE LISTING (4 sequences)

SEQ ID NO: 1 - DNA, 881 bp, Cladosporium fulvum
  CDS (3)..(779)
ttatggccgggggatacatggccggggaagactcacatcttcaccattcttctctaagcgctaggtctcggcgctcgaggccacaccaccccatccagcaatgccttcagcaagttccgcagcagcagcaccaagcaagcagcgagcatcagacatgcattgcgacagactggcaagactggcaagacgtctctggtgattggcaaggttttcaacagcaattgccgacatacgactggacagactgcaacatcgactacgatgaagcagctatgcaagccctgcaggatatgatcgacgcgtcagcatatcaagcgactgaagcaaacactgtctttagcacgacagatgccacatccacacctctgcagccaaatttactcttcgaccatcctcacgacatcgttccatcttaccaggatggtggccatctgagcacatctttaggagaagtacactccttcgctccagcaggcagcaacctatcatcgtcaagacctactgcgacgcctgactctactccggacaccacggcgcaccgtgtaggtggttctacgccgtcttctgttggcgctgactctcctggctcaggctcgagcaagatccgcaagagacaacgtaacaccgaagcggcccgtcgataccgccaaaggaagttggatagggcctcagagctcgaggaagctttggaagccatgggtaaagagcgagatgaacttaggctgaagcttgccaaggctgaaaccgaggctggtgtcttgcgagggttggttgggaagtagtactttggttgaaaatagtcatataattggaactgtattatggccatcatgtggcagacatgtctttccaaaaaaaaaaaaaaaaaaaaaaaaaaaaaa

SEQ ID NO: 2 - protein, 259 aa, Cladosporium fulvum
MAGGYMAGEDSHLHHSSLSARSRRSRPHHPIQQCLQQVPQQQHQASSEHQTCIATDWQDWQDVSGDWQGFQQQLPTYDWTDCNIDYDEAAMQALQDMIDASAYQATEANTVFSTTDATSTPLQPNLLFDHPHDIVPSYQDGGHLSTSLGEVHSFAPAGSNLSSSRPTATPDSTPDTTAHRVGGSTPSSVGADSPGSGSSKIRKRQRNTEAARRYRQRKLDRASELEEALEAMGKERDELRLKLAKAETEAGVLRGLVGK

SEQ ID NO: 3 - DNA, 20 bp, Artificial Sequence
  Description of Artificial Sequence primer
caatcacagtgttggcttgc

SEQ ID NO: 4 - DNA, 19 bp, Artificial Sequence
  Description of Artificial Sequence Primer
gaccctatgggctgtgttg